(19) United States
(12) Patent Application Publication
(10) Pub. No.: US 2021/0038811 A1
(43) Pub. Date: Feb. 11, 2021

(54) DEVICE FOR DELIVERY OF A LOCAL ANESTHETIC AGENT TO AN INCISION SITE DURING A SURGICAL PROCEDURE

(71) Applicant: TransEnterix Surgical, Inc., Morrisville, NC (US)

(72) Inventor: Matthew Robert Penny, Holly Springs, NC (US)

(21) Appl. No.: 16/932,678

(22) Filed: Jul. 17, 2020

Related U.S. Application Data
(60) Provisional application No. 62/874,990, filed on Jul. 17, 2019.

(51) Int. Cl. A61M 5/178; A61M 19/00

(52) U.S. Cl. CPC A61M 5/178; A61M 2202/048; A61M 19/00

(57) ABSTRACT

A drug delivery trocar is configured to deliver medication at an incision site during surgery so as to reduce post-surgical incisional pain. The trocar includes an elongate tube having a lumen, and a fluid reservoir attached or attachable to the lumen.

This application claims priority to U.S. Provisional Application No. 62/874990, filed Jul. 17, 2019, which is fully incorporated herein by reference.

### FIELD OF THE INVENTION

This invention relates to devices and methods for providing drug delivery to breached tissue surrounding trocars during surgery.

### BACKGROUND

Trocars are currently used during laparoscopic surgery to provide entry points for surgical instruments and surgical materials into the patient abdomen. Often, these entry points become sources of post-operative pain due to the incision and forces applied at the incision during surgery. In fact for most patients, the primary source of pain after surgery is pain at the incision site. Sometimes the pain is treated with opioids or other pain medication that may lead to addiction and other medical issues. Alternatively, some hospitals are utilizing medications such as Pivacain (Bupivacaine Hydrochloride), Exparel, etc. as local anesthetics injected around the trocar incisions before, during and after surgery to minimize post-operative pain.

The concepts described herein are intended to facilitate the controlled delivery of such anesthetics and/or other medications (i.e. clotting agents, growth-aiding agents, infection-fighting agents, etc), via the trocar and/or attachments to the trocar.

### BRIEF DESCRIPTION OF THE DRAWINGS

FIG. 1 is a cross-sectional side view of a first embodiment of a trocar.

FIG. 2 is a cross-sectional side view of a second embodiment of a trocar.

FIG. 3 is a cross-sectional side view of a third embodiment of a trocar.

FIG. 4 is a cross-sectional side view of a fourth embodiment of a trocar.

FIG. 5 is a perspective view of a fifth embodiment of a trocar.

FIG. 6 is a perspective view of a sixth embodiment of a trocar.

### DETAILED DESCRIPTION

There are several delivery mechanisms available for delivering medications such as Pivacain (or other medications) at the site of a trocar. For the purposes of this disclosure, it is assumed that the medication will either be packaged with the trocar or the trocar attachment, or will be applied to the trocar or trocar attachment at the time of use. For packaging with the trocar or trocar attachment, the medication may be contained in a reservoir or integrated with another polymer or substance that may facilitate a timed release.

In either case, packaged with the device or applied at the time of use, the medication may be introduced directly through the trocar itself (via the CO2 line, for example), through a reservoir built into the trocar, or through a reservoir that attaches to the trocar, externally. For the purposes of this document, a reservoir may consist of a volume containing only the medication or a volume of material that is mixed with the medication that could include plastics, polymers or other agents that modify the medication physical properties to facilitate a desired result such as a timed release. The following embodiments, which are depicted on the below chart, will describe each of these introduction methods in further detail.

### Delivery Through Trocar via External Reservoir

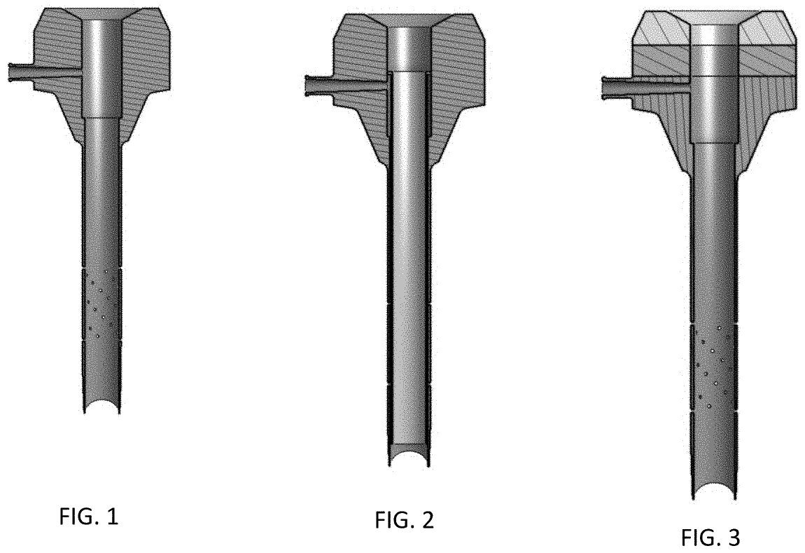

The first embodiment is comprised of an external reservoir for the medication intended to be delivered and the trocar, through which the delivery is executed. The trocar can consist of one or more lumens through which surgical instrumentation, and the medication may be delivered. A single lumen example may have more than one entry and exit point along its length. The surgical instrumentation may only be able to utilize one of the entry locations and one of the exit locations, typically in line with the axis of the single lumen, but not required. The medication may utilize one or more entry points and one or more exits. The most basic example can be seen in FIG. 1, where the medication would enter the trocar through the luer fitting, travel along the lumen with the surgical instrument and may exit the shaft at the distal end with the surgical instrument, as well as via perforations or holes positioned strategically along the lumen wall to deliver medication to the tissue surrounding the trocar shaft.

In FIG. 1, the medication reservoir may be a separate component that is attached to the luer port, integral to the pneumo tubing, or attached at the proximal end of the pneumo line. The release of medication from the reservoir may be controlled via the pump delivering gas to the abdominal site, via its own integral pumping mechanism or controlled by some external influence such as being manually actuated, or even tied to the physical movements of the surgical instrument engaged with the trocar lumen.

Potential Actuation Mechanisms:
Integration of reservoir into the pneumatic pump could enable the medication to be pushed via CO2 or potentially aerosolized with the CO2, if so desired. The mixture of the medication and CO2 could be controlled via software and a valve system to change the concentration and the airflow.
A reservoir within the line or at the interface between the CO2 line and the trocar could also facilitate an aerosolized mixture of CO2 and medication, or a pressure differential that could dispense liquid medication into the trocar lumen.
A reservoir within the line or at the interface between the CO2 line and the trocar could be manually actuated (i.e. squeezed) by a user to deliver a bolus of medication at any desired interval. The mechanism could be designed to only deliver a metered dose by having an internal valve mechanism between a primary reservoir and a dosage reservoir. This mixture could enable aerosolized or liquid dispensed medication.
A reservoir and/or valve system integrated or closely associated with the head of the trocar could be used to dispense medication based on the movement of surgical instrument shafts inside of the trocar lumen. For example, a compressible reservoir surrounding the trocar lumen, in the trocar head, but distal to the trocar seal, could dispense medication as the instrument shaft applies force to the reservoir. In particular, side to side motion of the instrument and/or axial motion of the instrument could be used to force fluid to exit the reservoir and migrate through the trocar lumen.

The above dispensing mechanisms and reservoir locations could also be utilized in trocars having more than one lumen, where the primary lumen is utilized for surgical instrument passage and the secondary lumen(s) are intended to facilitate transfer of the medication to the target sites.

A simple example of this concept may be a tube-in-a-tube, as shown in FIG. 2. In this embodiment, the innermost shaft is intended to guide the instrument, while the external shaft and the interstitial space allows medication to flow freely. The external shaft is perforated at the distances where it is anticipated that the medication should be delivered. In this concept, the lumen created by the interstitial space between tubes may not extend completely through the distal end of the trocar, thus preventing the medication from exiting the trocar into the abdomen and forcing it to interact with the tissue surrounding the trocar. It should be understood that, while perforations are illustrated in both of the previous embodiments, each embodiment can have any number or shape or distribution of openings or orifices to facilitate medication delivery to a target tissue. Additionally, external markings to indicate depth of the orifices may be helpful in setting the trocar to the proper position relative to the thickness of the fascial tissue.

The third embodiment for an attachment to the trocar consists of a medication reservoir, pre-filled or filled at the time of use, that can be attached to the head of the trocar itself. For some trocars, including those from Ethicon Endosurgery (e.g. the Endopath Exel Trocar), the trocar shaft seal can be removed from the trocar shaft as a means to remove large specimen or insert sharp objects that could damage the seal. For trocars of this type, in particular, a medication reservoir may be installed between the trocar shaft and the removable housing that includes the trocar shaft seal. FIG. 3 illustrates a trocar having a removable reservoir that is installed in the path of the surgical instrument shaft.

In the embodiment discussed above, the delivery actuation mechanisms could also include leveraging the pressure from the CO2 pneumoperitoneum line, manual activation and/or activation via pressure applied through the surgical instrument shaft. As with the previous embodiment, the reservoir attachment could deliver the medication via the primary lumen, or through secondary lumen(s) to the target tissue.

### Delivery through Trocar via Integrated Reservoir

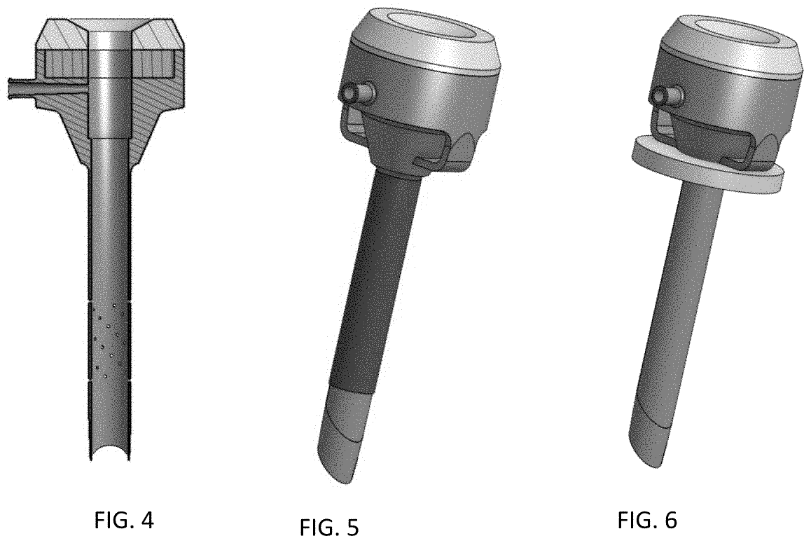

In this embodiment, shown in FIG. 4, the medication reservoir exists within the trocar and can be pre-loaded with the medication or filled up at the time of use. This reservoir could be proximal or distal to the trocar seal, but distal to the seal would be preferred such that the delivery pathway doesn't require a secondary seal for pneumoperitoneum. The following image illustrates an integral medication reservoir.

As with the previously described reservoir attachment, the integral reservoir could be pre-filled or filled at the time of use and could also utilize the same attachment mechanisms. The primary difference is that this embodiment is built directly into a custom trocar, whereas the reservoir attachment is intended for use with other off-the-shelf (OTS) trocars.

### Delivery External to the Trocar via External Reservoirs

The next embodiment consists of a reservoir sleeve that is engaged with the external shaft of the trocar. The sleeve is captured between the trocar shaft and the fascial wall and may dispense medications in response to forces applied to the trocar or manually actuated by squeezing an accessible reservoir, for example. The sleeve may also include an exposed luer fitting such that additional fluid may be added during the procedure without removing the trocar. See FIG. 5.

A final last embodiment includes a reservoir disc that is captured between the abdominal wall and the trocar head. This disc may be a compressible material, such as foam, that can be immersed or injected with the medication prior to, or during the procedure. As with other reservoirs detailed in this document, the medication may be dispensed by manual compression (i.e. force applied to the head of the trocar) or as a response to forces applied by the instrument at the trocar site. See FIG. 7.

While the reservoir disc may not provide direct access to the breached fascial tissue, this concept will be the least expensive option to pursue in addition to being the most flexible for various trocar diameters and manufacturers. In particular the foam may be pre-slit or die-cut such that the center can be dilated with any trocar shaft. Additionally, the foam could just allow the trocar to cut its own channel through the reservoir.

Other options for delivering anesthetic agent might include delivery of nanoparticles into, or on, the tissue at the incision site in order to deliver a slower-release agent to the site, giving longer lasting relief to the patient.

What is claimed is:

1. A drug delivery trocar comprising:
an elongate tube having a lumen;
a fluid reservoir attached or attachable to the lumen.
